(19) United States
(12) Patent Application Publication
(10) Pub. No.: US 2024/0189120 A1
(43) Pub. Date: Jun. 13, 2024

(54) PROSTHESIS SOCKET VACUUM SUCTION STRUCTURE

(71) Applicant: Ken Dall Enterprise Co., Ltd., New Taipei City (TW)

(72) Inventors: Chia-Pao CHENG, New Taipei City (TW); Hsiang-Ming WU, New Taipei City (TW)

(21) Appl. No.: 18/512,037

(22) Filed: Nov. 17, 2023

(30) Foreign Application Priority Data
Dec. 8, 2022 (CN) 202223291643.5

(51) Int. Cl. A61F 2/74; A61F 2/66

(52) U.S. Cl. CPC A61F 2/748; A61F 2/6607; A61F 2002/805

(57) ABSTRACT

A prosthesis socket vacuum suction structure includes a fluid chamber and a fluid supply compartment formed in an interior of an ankle pressure cylinder. The fluid chamber is divided by a piston to form upper and fluid chambers. A fluid-chamber channel is connected from the fluid supply compartment to the upper fluid chamber. A diversion channel is connected from the fluid-chamber channel to the lower fluid chamber. An angle of the ankle pressure cylinder oscillates through displacement, and the piston is driven to regulate interior fluid pressures of the upper and lower fluid chambers to realize balancing and cushioning. Further, an air chamber is arranged in the interior of the ankle pressure cylinder so as to reduce the size and also to simultaneously regulate an interior pressure of a socket through operation of the air chamber in combination with displacement of the user.

### BACKGROUND OF THE INVENTION

### (a) Technical Field of the Invention

The present invention relates to a prosthesis socket vacuum suction structure, which, through an angle of oscillation induced in the course of walking of a user, makes fluid pressure of a fluid chamber to displace between an upper fluid chamber and a lower fluid chamber for achieving balance of pressure in order to fulfill cushioning of pressure, and to cause, during walking, an air chamber to draw air from a socket so as to keep tightness between a residual limb stump of the user and the socket in a better state.

### (b) Description of the Prior Art

Prostheses, such as knee joints and ankle joints, which are currently available in the market include a mechanism that is of a pneumatic or hydraulic arrangement. Although the effect of shock absorption is relatively good, yet the user needs to arrange a socket to collaborate with the residual limb stump and lining to improve tightness therebetween, and as such, an air sucking assembly, which can be manually or electrically operated, to further evacuate the air from the socket for better tightness and a more comfortable contact condition in order to improve a suction-holding effect between the prosthesis and the residual limb stump of the user.

However, for the above-described known air sucking assemblies, whether a pneumatic mechanism or a hydraulic mechanism, to be used in a knee joint, most of them are independently mounted outside of the knee joint, so that the size of the entire knee joint cannot be effectively reduced. As such, for the residual limb stump of the user, the perception of wearing is, to some extents, burdensome.

The present inventor has proposed, in previously filed U.S. patent application Ser. No. 13/920,027, which discloses an improved structure for prosthesis ankle joint and socket vacuum suction, an improvement in which the air sucking assembly is arranged in a main body of a knee joint, so that with the posture of the user in walking, tightness for suction-attaching to the socket can be effectively driven and regulated to improve the tedious operation of repeated adjustment. Although the structure so improved has an advantage of arranging the entirety of the air sucking assembly in the interior of the knee joint, it is still impossible to greatly reduce the overall size. Thus, additional studies and mockups have been made to provide a perfect solution.

### SUMMARY OF THE INVENTION

The present invention relates to a prosthesis socket vacuum suction structure, which comprises: an ankle pressure cylinder having an interior in which a fluid chamber, a fluid supply compartment, and an air chamber are arranged. A piston is arranged in an interior of the fluid chamber to divide the fluid chamber into an upper fluid chamber and a lower fluid chamber. A fluid-chamber channel is formed in a top end of the fluid supply compartment and connected to and in communication with the upper fluid chamber. A diversion channel is connected from a middle of the fluid-chamber channel to the lower fluid chamber. A socket assembly is connected to an upper portion of the ankle pressure cylinder. A prosthesis sole is connected to a lower portion of the ankle pressure cylinder.

An angle of the ankle pressure cylinder is changeable, as being oscillated, through displacement, so that the piston regulates interior fluid pressures of the upper fluid chamber and the lower fluid chamber to achieve balancing and cushioning. In an alternative embodiment, an air chamber is formed in the interior of the ankle pressure cylinder to collaborate with a socket assembly as a function for connecting a socket, so that in addition to the essential fluid pressure balance, it is also possible to simultaneously fulfill vacuum regulation.

### BRIEF DESCRIPTION OF THE DRAWINGS

The attached drawings that are applied in combination with the disclosure and constitute a part of the disclosure illustrate embodiments of the present invention, and will be applied, in combination with the disclosure, to explain the principle of the present invention.

FIG. 1 is a perspective view of the present invention.

FIG. 2 is a schematic view, in a sectioned form, illustrating action of the present invention based on FIG. 1.

FIG. 3 is a schematic view, in a sectioned form, illustrating successive action of the present invention based on FIG. 2.

FIG. 4 is a schematic view, in a sectioned form, illustrating successive action of the present invention based on FIG. 3.

FIG. 5 is a perspective view of another embodiment of the present invention.

FIG. 6 is a left side view of said another embodiment of the present invention based on FIG. 5.

FIG. 7 is a right side view of said another embodiment of the present invention based on FIG. 5.

FIG. 8 is a schematic view illustrating action of said another embodiment of the present invention in a state of being ready to move.

FIG. 9 is a schematic view, in a sectioned form, illustrating a portion of said another embodiment of the present invention based on FIG. 8.

FIG. 10 is a schematic view illustrating action of said another embodiment of the present invention in a course of movement.

FIG. 11 is a schematic view, in a sectioned form, illustrating a portion of said another embodiment of the present invention based on FIG. 10.

FIG. 12 is a schematic view illustrating action of said another embodiment of the present invention in a state of completing movement.

FIG. 13 is a schematic view, in a sectioned form, illustrating a portion of said another embodiment of the present invention based on FIG. 12.

### DETAILED DESCRIPTION OF THE PREFERRED EMBODIMENTS

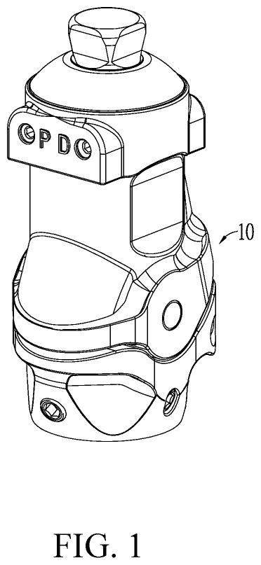

A detailed description of the present invention will be provided below with reference to preferred feasible embodiments, in combination with the attached drawings, for better understanding of the present invention. Referring to FIGS. 1-4, the present invention provides a prosthesis socket vacuum suction structure, which comprises: an ankle pressure cylinder 10, which comprises a fluid chamber 11 and a fluid supply compartment 12 formed in an interior thereof. A piston 111 is arranged in an interior of the fluid chamber 11, and the piston 111 divides the interior such that an upper portion of the fluid chamber 11 defines an upper fluid chamber 112 and a lower portion of the fluid chamber 11 defines a lower fluid chamber 113.

A fluid-chamber channel 121 is formed in a top end of the fluid supply compartment 12 and is connected to and in communication with the upper fluid chamber 112. A diversion channel 122 is connected from a middle of the fluid-chamber channel 121 to the lower fluid chamber 113. The fluid supply compartment 12 is provided, in an interior thereof, with a one-way valve so that fluid contained therein is only allowed flow out. A socket assembly 20 is connected to an upper portion of the ankle pressure cylinder 10, and a prosthesis sole 30 is connected to a lower portion of the ankle pressure cylinder 10.

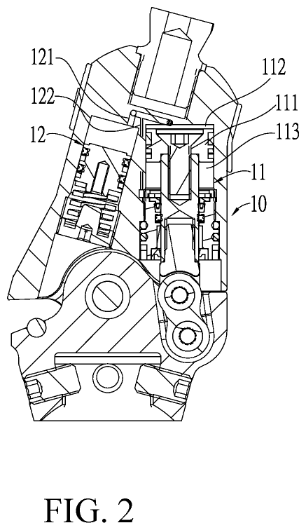

The fluid chamber 11 and the fluid supply compartment 12 are both arranged in the interior of the ankle pressure cylinder 10, so that an overall size is greatly reduced, and also, due to rearrangement with respect to distance, location, and message channel, there is no need to involve three-dimensional pipeline arrangement for control one-way or two-way flowing, and as such, improvement to a certain extent is achieved as compared to the previous patent applications of the present inventor. An angle of the ankle pressure cylinder 10 is changed, as being oscillated, through displacement as depicted in FIGS. 2-4, which illustrate a cycle of walking. FIG. 2 shows stepping forward, wherein the fluid contained in an interior of the upper fluid chamber 112 passes through the fluid-chamber channel 121 and the diversion channel 122 into the lower fluid chamber 113.

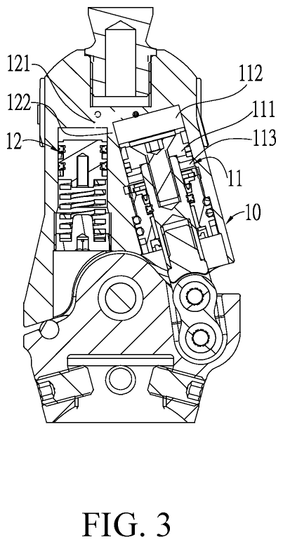

FIG. 3 shows an upright state, wherein the fluid contained in the lower fluid chamber 113 is subjected to a pressure induced by downward displacement of the piston 111 to have a portion of the fluid moving from the diversion channel 122 through the fluid-chamber channel 121 into the upper fluid chamber 112.

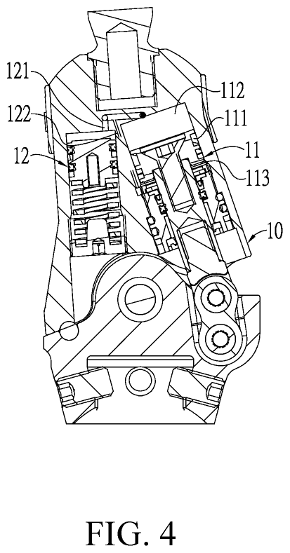

FIG. 4 shows rearward inclining, wherein the upper fluid chamber 112 is completely filled with the fluid. In case that the fluid is depleted with time and becomes insufficient, due to the arrangement of the one-way valve in the fluid supply compartment 12, the fluid can be automatically supplied, due to pressure, from the fluid-chamber channel 121 for supplementary in order to keep the best condition of the fluid between the fluid-chamber channel 121 and the diversion channel 122. The piston 111 is primarily for regulating the interior fluid pressures of the upper fluid chamber 112 and the lower fluid chamber 113 to realize balancing and cushioning.

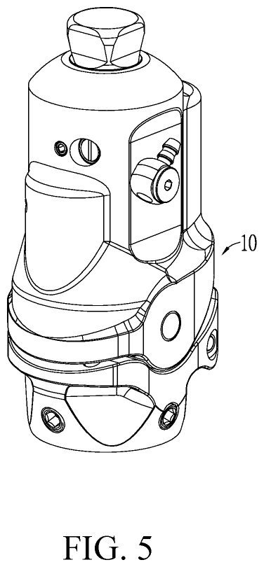

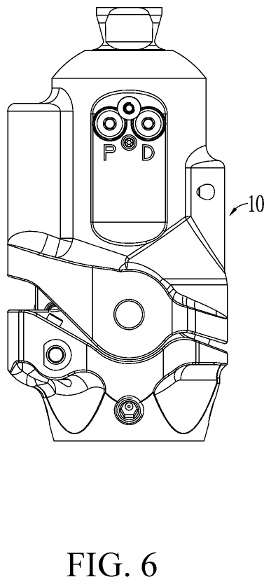

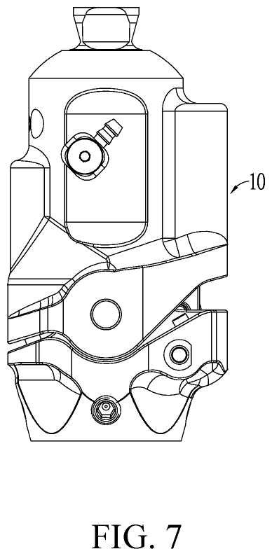

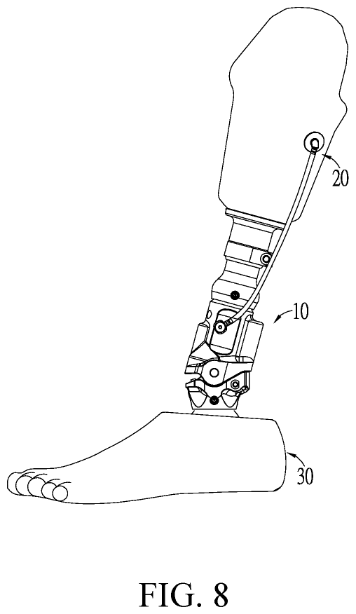

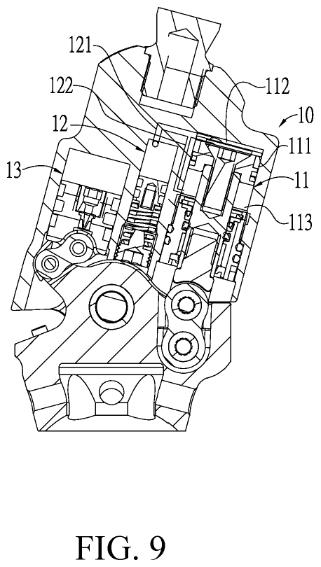

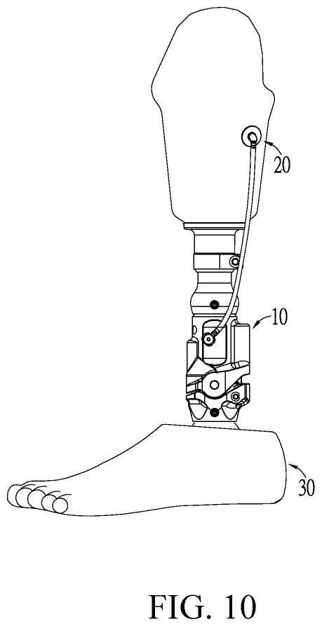

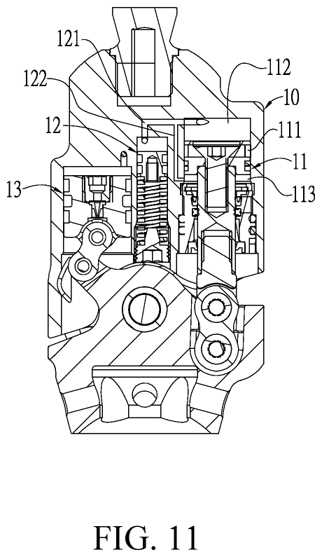

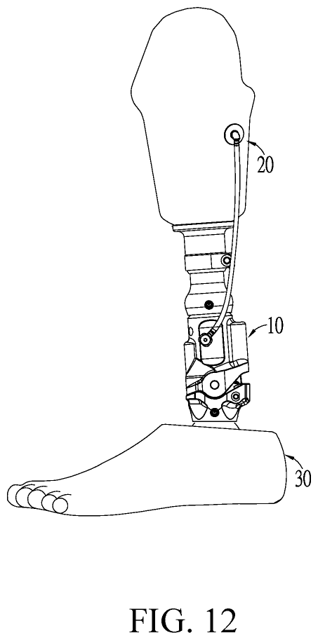

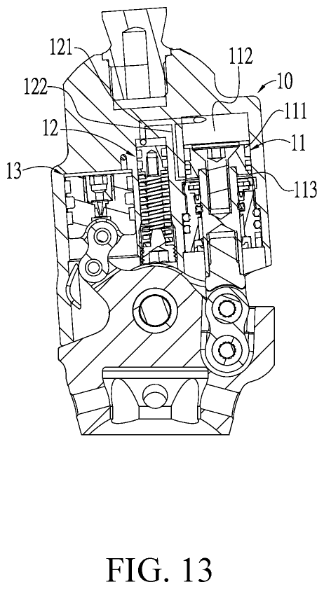

Referring to FIGS. 5-13, another embodiment of the present invention is shown, having a structure comprising: an ankle pressure cylinder 10, which comprises a fluid chamber 11, a fluid supply compartment 12, and an air chamber 13 formed in an interior thereof. A piston 111 is arranged in an interior of the fluid chamber 11, and the piston 111 divides the interior such that an upper portion of the fluid chamber 11 defines an upper fluid chamber 112 and a lower portion of the fluid chamber 11 defines a lower fluid chamber 113.

A fluid-chamber channel 121 is formed in a top end of the fluid supply compartment 12 and is connected to and in communication with the upper fluid chamber 112. A diversion channel 122 is connected from a middle of the fluid-chamber channel 121 to the lower fluid chamber 113. The fluid supply compartment 12 is provided, in an interior thereof, with a one-way valve so that fluid contained therein is only allowed flow out. A socket assembly 20 is connected to an upper portion of the ankle pressure cylinder 10, and a prosthesis sole 30 is connected to a lower portion of the ankle pressure cylinder 10. An angle of the ankle pressure cylinder 10 is changed, as being oscillated, through displacement, so that the piston 111 may regulate the interior fluid pressures of the upper fluid chamber 112 and the lower fluid chamber 113 to realize balancing and cushioning. Here, the fluid flowing between the fluid-chamber channel 121 and the diversion channel 122 is similar to what described above and thus repeated description will be omitted. The newly included air chamber 13 is provided for collaborating with the socket assembly 20 for vacuum pressure regulating to allow tightness of suction-attaching of the socket assembly 20 to a user to be kept at a preset level.

In summary, the present invention provides a prosthesis socket vacuum suction structure, in which the fluid chamber 11 and the fluid supply compartment 12 are arranged in the interior of the ankle pressure cylinder 10 and the fluid chamber 11 is divided by the piston 111 into the upper fluid chamber 112 and the lower fluid chamber 113, so that change of the ankle pressure cylinder 10 through displacement caused by walking of a user changes interior fluid pressures to achieve a balanced, pressure-stable condition, and further, an air chamber 13 is additionally included in the structure to be arranged in the interior of the ankle pressure cylinder 10 to collaborate with the socket assembly 20 for maintain tightness between a residual limb stump of a user and the socket during the course of walking, so as to realize automatic vacuum regulation and thus reduce discomfort of the user and make the size of the overall structure reduced.

I claim:

1. A prosthesis socket vacuum suction structure, comprising: an ankle pressure cylinder having an interior in which a fluid chamber and a fluid supply compartment are arranged, a piston being arranged in an interior of the fluid chamber respectively define an upper fluid chamber and a lower fluid chamber in an upper portion and a lower portion thereof, a fluid-chamber channel being formed in a top end of the fluid supply compartment and connected to and in communication with the upper fluid chamber, a diversion channel being connected from a middle of the fluid-chamber channel to the lower fluid chamber; a socket assembly, which is connected to an upper portion of the ankle pressure cylinder; and a prosthesis sole, which is connected to a lower portion of the ankle pressure cylinder, wherein an angle the ankle pressure cylinder is oscillated through displacement to allow the piston to regulate interior fluid pressures of the upper fluid chamber and the lower fluid chamber for balancing and cushioning.

2. The prosthesis socket vacuum suction structure according to claim 1, wherein the fluid supply compartment comprises a one-way flowing arrangement.
